(19) United States
(12) Patent Application Publication
(10) Pub. No.: US 2002/0045734 A1
(43) Pub. Date: Apr. 18, 2002

(54) Novel reovirus-derived proteins and uses therefor

(76) Inventor: Roy Duncan, Nova Scotia (CA)

Correspondence Address: SMART & BIGGAR, 900-55 Metcalfe Street, P.O. Box 2999, Station D, Ottawa, ON, K1P 5Y6, CA

(21) Appl. No.: 09/943,002

(22) Filed: Aug. 31, 2001

Related U.S. Application Data
(63) Continuation of application No. 08/965,708, filed on Nov. 7, 1997.

(51) Int. Cl. C07K 14/01; C12N 15/86; C12P 21/02; C12N 5/06

(52) U.S. Cl. 530/350; 435/325; 435/69.3; 435/320.1; 536/23.72

(57) ABSTRACT

In accordance with the present invention, viral proteins that are responsible for membrane fusion and syncytium formation induced by three different fusogenic orthoreoviruses, i.e., avian reoviruses (ARV), Nelson Bay virus (NBV), and Baboon Reovirus (BRV), have been identified. The genes encoding these proteins have been cloned and sequenced; functional analysis thereof indicates that expression of these proteins in transfected cells results in cell-cell fusion.

### REFERENCE TO RELATED APPLICATION

This is a continuation of Application Ser. No. 08/965,708, filed Nov. 7, 1997.

### FIELD OF THE INVENTION

The present invention relates to novel viral-derived proteins and uses thereof.

### BACKGROUND OF THE INVENTION

The integrity and function of eukaryotic cells is dependent on the presence of discrete membrane compartments, surrounded by impermeable lipid bilayers. The hydrophobic nature of these lipid bilayers presents an effective barrier to nearly all types of charged or polar molecules. The impermeability of the cell membrane is a confounding factor in the effective intracellular delivery of nucleic acids, proteins, and pharmacologic agents in both clinical and research applications and has lead to the development of liposome-based delivery systems (see, for example, Mannino, Biotechniques, 6:682-690 (1988); and Gao, Gene Ther., 2:710-722 (1995)).

The intent of liposome-based delivery systems is to encapsulate bioactive molecules inside lipid vesicles and to promote liposome-cell fusion to facilitate intracellular delivery. However, the polar lipid headgroups oriented on both surfaces of the lipid bilayer, along with an associated water layer, make spontaneous membrane fusion a thermodynamically unfavorable process. Yet cell-cell membrane fusion (as during sperm-egg fusion or muscle cell differentiation to myotube) and intracellular membrane fusion (as part of the vesicle transport system in cells) are essential cellular processes (White, Science, 258:917-924 (1992)).

In addition to natural cell-cell fusion, experimentally induced cell-cell fusion is also a valuable procedure for the production of heterokaryons for research purposes, as well as for commercial applications. Although various chemicals or lipids can be used to experimentally promote membrane fusion, these reagents usually exhibit cytotoxic effects (see, for example, Iwamoto et al., in Biol. Pharm. Bull. 19:860-863 (1996) and Mizuguchi et al., in Biochem. Biophys. Res. Commun., 218:402-407 (1996)). It is generally believed that membrane fusion under physiological conditions is protein-mediated, which has led to the development of proteoliposomes (i.e., liposomes containing proteins that promote membrane fusion) to promote more efficient liposome-cell fusion, with decreased cytotoxicity (see, for example, Cheng, Hum. Gene Ther. 7:275-282 (1996); Hara et al., Gene 159:167-174 (1995); and Findeis et al., Trends Biotechnol., 11:202-205 (1993)).

The choice of proteins to be used to enhance membrane fusion is limited by their availability. The only proteins conclusively shown to induce membrane fusion are those of the enveloped viruses. All enveloped viruses encode fusion proteins that are responsible for fusion of the viral envelope with the cell membrane. These viral fusion proteins are essential for successful infection of susceptible cells. Indeed, their mechanism of action serves as a paradigm for protein-mediated membrane fusion (see, for example, White, Ann. Rev. Physiol., 52:675-697 (1990); and White, Science, 258:917-924 (1992)).

Most enveloped virus fusion proteins are relatively large, multimeric, type I membrane proteins, as typified by the influenza virus HA protein, a low pH-activated fusion protein, and the Sendai virus F protein, which functions at neutral pH. The majority of the fusion protein is oriented on the external surface of the virion to facilitate interactions between the virus particle and the cell membrane. Fusion of the viral envelope with the cell membrane is mediated by an amphipathic alpha-helical region, referred to as a fusion peptide motif, that is part of the viral fusion protein. Recognition of the importance of fusion peptides in triggering membrane fusion has resulted in the use of small peptides that resemble fusion peptides to enhance liposome-cell fusion (see, for example, Muga et al., Biochemistry 33:4444-4448 (1994)).

Enveloped virus fusion proteins also trigger cell-cell fusion, resulting in the formation of polykaryons (syncytia). Synthesis of the viral fusion protein inside the infected cell results in transport of the fusion protein through the endoplasmic reticulum and Golgi transport system to the cell membrane, an essential step in the assembly and budding of infectious progeny virus particles from the infected cell (Petterson, Curr. Top. Micro. Immunol., 170:67-106 (1991)). The synthesis, transport, and folding of the fusion protein is facilitated by a variety of components, e.g., signal peptides to target the protein to the intracellular transport pathway, glycosylation signals for N-linked carbohydrate addition to the protein, and a transmembrane domain to anchor the protein in the cell membrane. The ability of enveloped virus fusion proteins to promote efficient membrane fusion has resulted in the use of these proteins in reconstituted proteoliposomes (virosomes) for protein-mediated enhanced liposome-cell fusion both in cell culture and in vivo (see, for example, Ramani et al., FEBS Lett., 404:164-168 (1997); Scheule et al., Am. J. Respir. Cell Mol. Biol., 13:330-343 (1995); and Grimaldi, Res. Virol., 146:289-293 (1995)).

Unlike enveloped viruses, the nonenveloped viruses generally do not encode fusion proteins since the absence of a viral membrane precludes entry mediated by membrane fusion. Because progeny virus particles of nonenveloped viruses do not need to acquire a lipid envelope, these viruses usually do not bud from infected cells but, rather, are released by cell lysis. As a result, nonenveloped viruses do not express fusion proteins on the surface of infected cells and, hence, do not induce syncytium formation. The only exception to this situation occurs with selected members of the family Reoviridae (see Duncan et al., Virology, 212:752-756 (1995), and references therein), a family of nonenveloped viruses containing segmented double-stranded RNA (dsRNA) genomes (see, for example, Nibert et al., Reoviruses and their replication, In: Fundamental Virology, 3rd Edition, B. N. Fields, D. M. Knipe and P. M. Howley (Eds), Lippincott-Raven Press, NY (1996)).

It would be desirable, therefor, to identify additional proteins which induce membrane fusion and to develop new methodologies for inducing membrane fusion. These and other needs are satisfied by the present invention, as will become apparent upon review of the specification and appended claims.

### BRIEF DESCRIPTION OF THE INVENTION

The genus Orthoreovirus contains two distinct subgroups, the avian and the mammalian reoviruses. Unlike their mammalian counterparts, the avian reoviruses (ARV) are all fusogenic and induce rapid and extensive cell-cell fusion, resulting in syncytium formation in infected cell cultures (see Robertson and Wilcox, Vet. Bull., 56:726-733 (1986)). In addition to ARV, there are two atypical mammalian reoviruses that induce cell-cell fusion; one was isolated from a flying fox and is named Nelson Bay virus (NBV) (see Gard and Compans, J. Virol., 6:100-106 (1970)) while the other was isolated from a baboon and is referred to as Baboon Reovirus (BRV) (see Duncan et al., Virology, 212:752-756 (1995)).

In accordance with the present invention, the viral proteins that are responsible for membrane fusion and syncytium formation induced by these three different fusogenic orthoreoviruses have been identified. The genes encoding these proteins have been cloned and sequenced; functional analysis thereof indicates that expression of these proteins in transfected cells results in cell-cell fusion.

These atypical nonenveloped viral fusion proteins are unrelated to any previously identified fusion proteins and represent a new family of viral fusion proteins, the first identified from a nonenveloped virus. Sequence analysis of these atypical fusion proteins indicates several unique structural features and suggest their utility as attractive agents for the intracellular delivery of various compounds via protein-mediated liposome-cell fusion and for use in promoting cell-cell fusion. The structural and functional characterization of invention reovirus fusion proteins is described herein.

### BRIEF DESCRIPTION OF THE FIGURES

FIG. 1 collectively presents schematic diagrams of reovirus fusion protein-encoding genome segments. Thus, FIG. 1A presents segments from avian reovirus (ARV); FIG. 1B presents segments from Nelson bay virus (NBV); and FIG. 1C presents segments from Baboon Reovirus (BRV).

FIG. 2 presents aligned sequences of the P11 proteins of ARV and NBV. Dots indicate small insertions to maintain the alignment. The consensus sequence indicates positions where all three sequences agree; dashes indicate that no consensus exists at that location. The predicted transmembrane domain is overlined and labeled. Asterixes indicate the locations of conserved cysteine residues, while + symbols indicate conserved basic amino acid residues.

FIG. 3 presents the amino acid sequence of the BRV P15a fusion protein. The predicted transmembrane domain is overlined and labeled. The cluster of positively charged amino acids adjacent to the transmembrane is labeled with a + symbol.

### DETAILED DESCRIPTION OF THE INVENTION

In accordance with the present invention, there are provided proteins characterized as:

having a molecular weight of about 11,000,

having less than about 100 amino acid residues,

having one transmembrane domain,

having a relatively small intracellular domain,

having a relatively small extracellular domain,

wherein said extracellular domain contains an amphipathic alpha helix motif, and

being relatively non-immunogenic,

wherein said proteins are further characterized as lacking:

signal peptide, and

N-linked glycosylation signals.

Exemplary proteins embraced by the above-described profile of properties include proteins having an amino acid sequence substantially the same as set forth in SEQ ID NO:2, proteins having an amino acid sequence substantially the same as set forth in SEQ ID NO:6, proteins having an amino acid sequence substantially the same as set forth in SEQ ID NO:8, and the like.

Presently preferred proteins embraced by the above-described profile of properties include proteins having the same amino acid sequence as set forth in SEQ ID NO:2, proteins having the same amino acid sequence as set forth in SEQ ID NO:6, proteins having the same amino acid sequence as set forth in SEQ ID NO:8, and the like.

In accordance with another aspect of the present invention, there are provided proteins characterized as:

having a molecular weight of about 15,000,

having less than about 150 amino acid residues,

having one transmembrane domain,

having one relatively small intracellular domain,

having a relatively small extracellular domain,

wherein said extracellular domain contains an alpha helix motif, and

being relatively non-immunogenic,

wherein said protein is further characterized as lacking:

signal peptide, and

N-linked glycosylation signals.

Exemplary proteins embraced by the above-described profile of properties include proteins having an amino acid sequence substantially the same as set forth in SEQ ID NO:10. Presently preferred proteins embraced by the above-described profile of properties include proteins having the same amino acid sequence as set forth in SEQ ID NO:10.

In accordance with yet another embodiment of the present invention, there are provided antibodies generated against the above-described fusion proteins. Such antibodies can be employed for diagnostic applications, therapeutic applications, and the like. Preferably, for therapeutic applications, the antibodies employed will be monoclonal antibodies.

The above-described antibodies can be prepared employing standard techniques, as are well known to those of skill in the art, using the invention fusion proteins, or fragments thereof, as antigens for antibody production. Antibodies of the present invention are typically produced by immunizing a mammal with an inoculum containing an invention fusion protein or polypeptide fragment thereof and thereby inducing in the mammal antibody molecules having immunospecificity for such fusion protein or polypeptide fragment thereof.

For example, antibodies raised in rabbits against a synthetic peptide recognize the synthetic peptide and the invention fusion protein on an equimolar basis, and, preferably, they are capable of inhibiting the activity of the native protein. Antibodies to such fusion proteins may be obtained, for example, by immunizing three month old male and female white New Zealand rabbits with the synthetic peptide to which Tyr has been added at the C-terminus in order to couple it, as an antigen, to BSA by a bisdiazotized benzidine (BDB) linkage by reaction for 2 hours at 4° C. The reaction mixture is dialyzed to remove low molecular weight material, and the retentate is frozen in liquid nitrogen and stored at −20° C. Animals are immunized with the equivalent of 1 mg of the peptide antigen according to the procedure of Benoit et al. P.N.A.S. USA, 79, 917-921 (1982). At four week intervals, the animals are boosted by injections of 200 μg of the antigen and bled ten to fourteen days later. After the third boost, antiserum is examined for its capacity to bind radioiodinated antigen peptide prepared by the chloramine-T method and then purified by CMC-ion exchange column chromatography. The antibody molecules are then collected from the mammal and isolated to the extent desired by well known techniques such as, for example, by using DEAE-Sephadex to obtain the IgG fraction.

To enhance the specificity of the antibody, the antibodies may be purified by immunoaffinity chromatography using solid phase-affixed immunizing polypeptide. The antibody is contacted with the solid phase-affixed immunizing polypeptide for a period of time sufficient for the polypeptide to immunoreact with the antibody molecules to form a solid phase-affixed immunocomplex. The bound antibodies are separated from the complex by standard techniques.

A radioimmunoassay is established with the antisera and serum from subsequent bleeds from the same rabbits. The native protein is recognized by the antibodies on an equimolar basis as compared to the synthetic peptide antigen.

The antibody so produced can be used, inter alia, in diagnostic methods and systems to detect the level of invention fusion protein present in a test sample. The anti-fusion protein antibodies can also be used for the immunoaffinity or affinity chromatography purification of such fusion proteins. In addition, an anti-fusion protein antibody according to the present invention can be used in therapeutic methods, e.g., blocking the occurrence of undesired fusion processes.

In accordance with yet another aspect of the present invention, there are provided nucleic acids encoding the above-described proteins, optionally operatively associated with a promoter. In addition, such nucleic acid molecules or fragments thereof can be labeled with a readily detectable substituent and used as hybridization probes for assaying for the presence and/or amount of genes encoding invention proteins or mRNA transcripts thereof in a given sample. The nucleic acid molecules described herein, and fragments thereof, are also useful as primers and/or templates in a PCR reaction for amplifying genes encoding the fusion proteins described herein.

Exemplary isolated nucleic acids contemplated for use in the practice of the present invention include nucleic acids having a contiguous nucleotide sequence substantially the same as:

nucleotides 25-1607 of SEQ ID NO:1,

nucleotides 25-1607 of SEQ ID NO:5,

nucleotides 27-1579 of SEQ ID NO:7,

nucleotides 25-832 of SEQ ID NO:9, or

variations thereof which encode the same amino acid sequence, but employ different codons for some of the amino acids, or splice variant nucleotide sequences thereof.

Presently preferred isolated and purified nucleic acids, or functional fragments thereof contemplated according to the invention are nucleic acids encoding the above-described proteins, e.g.,

(a) DNA encoding the amino acid sequence set forth in SEQ ID NO:2, SEQ ID NO:6, SEQ ID NO:8 or SEQ ID NO:10, or

(b) DNA that hybridizes to the DNA of (a) under moderately stringent conditions, wherein said DNA encodes biologically active fusion protein, or

(c) DNA degenerate with respect to either (a) or (b) above, wherein said DNA encodes biologically active fusion protein.

As employed herein, the term “contiguous nucleotide sequence substantially the same as” refers to DNA having sufficient homology to the reference polynucleotide, such that it will hybridize to the reference nucleotide under typical stringency conditions employed by those of skill in the art. In one embodiment, DNA having substantially the same nucleotide sequence as the reference nucleotide encodes substantially the same amino acid sequence of SEQ ID NOs:2, 6, 8 or 10. In another embodiment, DNA having “a contiguous nucleotide sequence substantially the same as” has at least 60% homology with respect to the nucleotide sequence of the reference DNA fragment with which the subject DNA is being compared. In a preferred embodiment, the DNA has at least 70%, more preferably 80%, homology to the comparative nucleotide sequence; with greater than about 90% homology being especially preferred.

Promoters contemplated for use herein include inducible (e.g., minimal CMV promoter, minimal TK promoter, modified MMLV LTR), constitutive (e.g., chicken β-actin promoter, MMLV LTR (non-modified), DHFR), and/or tissue specific promoters.

Inducible promoters contemplated for use in the practice of the present invention comprise transcription regulatory regions that function maximally to promote transcription of mRNA under inducing conditions. Examples of suitable inducible promoters include DNA sequences corresponding to: the E. coli lac operator responsive to IPTG (see Nakamura et al., Cell, 18:1109-1117, 1979); the metallothionein promoter metal-regulatory-elements responsive to heavy-metal (e.g., zinc) induction (see Evans et al., U.S. Pat. No. 4,870,009), the phage T71ac promoter responsive to IPTG (see Studier et al., Meth. Enzymol., 185: 60-89, 1990; and U.S. Pat. No. 4,952,496), the heat-shock promoter; the TK minimal promoter; the CMV minimal promoter; a synthetic promoter; and the like.

Exemplary constitutive promoters contemplated for use in the practice of the present invention include the CMV promoter, the SV40 promoter, the DHFR promoter, the mouse mammary tumor virus (MMTV) steroid-inducible promoter, Moloney murine leukemia virus (MMLV) promoter, elongation factor 1α (EF1α) promoter, albumin promoter, APO A1 promoter, cyclic AMP dependent kinase II (CaMKII) promoter, keratin promoter, CD3 promoter, immunoglobulin light or heavy chain promoters, neurofilament promoter, neuron specific enolase promoter, L7 promoter, CD2 promoter, myosin light chain kinase promoter, HOX gene promoter, thymidine kinase (TK) promoter, RNA Pol II promoter, MYOD promoter, MYF5 promoter, phosphoglycerokinase (PGK) promoter, Stf1 promoter, Low Density Lipoprotein (LDL) promoter, and the like.

In accordance with a further embodiment of the present invention, optionally labeled cDNAs encoding invention fusion proteins, or fragments thereof, can be employed to probe library(ies) (e.g., cDNA, genomic, and the like) for additional sequences encoding novel fusion proteins. Such screening is typically initially carried out under low-stringency conditions, which comprise a temperature of less than about 42° C., a formamide concentration of less than about 50%, and a moderate to low salt concentration. Presently preferred screening conditions comprise a temperature of about 37° C., a formamide concentration of about 20%, and a salt concentration of about 5× standard saline citrate (SSC; 20× SSC contains 3 M sodium chloride, 0.3 M sodium citrate, pH 7.0). Such conditions will allow the identification of sequences which have a substantial degree of similarity with the probe sequence, without requiring perfect homology for the identification of a stable hybrid. The phrase “substantial similarity” refers to sequences which share at least 50% homology. Preferably, hybridization conditions will be selected which allow the identification of sequences having at least 70% homology with the probe, while discriminating against sequences which have a lower degree of homology with the probe.

As used herein, a nucleic acid “probe” is single-stranded DNA or RNA, or analogues thereof, that has a sequence of nucleotides that includes at least 14, preferably at least 20, more preferably at least 50, contiguous bases that are the same as (or the complement of) any 14 or more contiguous bases set forth in any of SEQ ID NOs:1, 5, 7 or 9. Probes may be labeled by methods well-known in the art, as described hereinafter, and used in various diagnostic kits.

As used herein, the terms “label” and “indicating means” in their various grammatical forms refer to single atoms and molecules that are either directly or indirectly involved in the production of a detectable signal to indicate the presence of a complex. Any label or indicating means can be linked to or incorporated in a nucleic acid probe, an expressed protein, polypeptide fragment, or antibody molecule that is part of an antibody or monoclonal antibody composition of the present invention, or used separately. These atoms or molecules can be used alone or in conjunction with additional reagents. Such labels are themselves well-known in clinical diagnostic chemistry.

The labeling means can be a fluorescent labeling agent that chemically binds to antibodies or antigens without denaturing them to form a fluorochrome (dye) that is a useful immunofluorescent tracer. Suitable fluorescent labeling agents are fluorochromes such as fluorescein isocyanate (FIC), fluorescein isothiocyanate (FITC), 5-dimethylamine-1-naphthalenesulfonyl chloride (DANSC), tetramethylrhodamine isothiocyanate (TRITC), lissamine, rhodamine 8200 sulphonyl chloride (RB-200-SC), and the like. A description of immunofluorescence analysis techniques is found in DeLuca, “Immunofluorescence Analysis”, in Antibody As a Tool, Marchalonis et al., Eds., John Wiley & Sons, Ltd., pp. 189-231 (1982), which is incorporated herein by reference.

In preferred embodiments, the indicating group is an enzyme, such as horseradish peroxidase (HRP), glucose oxidase, and the like. In such cases where the principal indicating group is an enzyme, additional reagents are required to visualize the fact that a receptor-ligand complex (immunoreactant) has formed. Such additional reagents for HRP include hydrogen peroxide and an oxidation dye precursor such as diaminobenzidine. An additional reagent useful with glucose oxidase is 2,2′-azino-di-(3-ethyl-benzthiazoline-G-sulfonic acid) (ABTS).

Radioactive elements are also useful labeling agents and are used illustratively herein. An exemplary radiolabeling agent is a radioactive element that produces gamma ray emissions. Elements which emit gamma rays, such as ¹²⁴I, ¹²⁵I, ¹²⁶I, ¹³¹I and ⁵¹Cr, represent one class of radioactive element indicating groups. Particularly preferred is ¹²⁵I. Another group of useful labeling means are those elements such as ¹¹C, ¹⁸F, ¹⁵⁰O and ¹³N which emit positrons. The positrons so emitted produce gamma rays upon encounters with electrons present in the animal's body. Also useful is a beta emitter, such as ³²p, ¹¹¹In or ³H.

The linking of labels to substrate, i.e., labeling of nucleic acid probes, antibodies, polypeptides, and proteins, is well known in the art. For instance, antibody molecules produced by a hybridoma can be labeled by metabolic incorporation of radioisotope-containing amino acids provided as a component in the culture medium. See, for example, Galfre et al., Meth. Enzymol., 73:3-46 (1981). The techniques of protein conjugation or coupling through activated functional groups are particularly applicable. See, for example, Aurameas et al., Scand. J. Immunol., Vol. 8, Suppl. 7:7-23 (1978), Rodwell et al., Biotech., 3:889-894 (1984), and U.S. Pat. No. 4,493,795.

In accordance with still another aspect of the present invention, there are provided cells containing the above-described proteins.

In accordance with a still further aspect of the present invention, there are provided cells containing the above-described nucleic acids.

In accordance with yet another aspect of the present invention, there are provided liposomes containing the above-described proteins and/or nucleic acids. As is well known in the art, liposomes are sealed, usually spherical vesicles composed of lipid membrane bilayers enclosing a central aqueous compartment. Liposomes can be used for the delivery of nucleic acids and other biological materials to mammalian cells. See, for example, New, in Molecular Biology and Biotechnology, pp 514-516, R. A. Meyers, Ed., VCH Publishers, NY (1995), and references cited therein.

In accordance with still another aspect of the present invention, there are provided methods for the production of the above-described proteins, said methods comprising expressing nucleic acid encoding said protein in a suitable host.

In accordance with yet another aspect of the present invention there are provided methods to promote membrane fusion, said methods comprising contacting the membranes to be fused with an effective amount of the above-described proteins.

Membranes contemplated for fusion in accordance with the present invention include cell membranes, liposome membranes, proteoliposome membranes, and the like.

In accordance with a still further embodiment of the present invention, there are provided methods for the production of heterokaryons, such as B cell or T cell hybridoma cells useful for the production of monoclonal antibodies, cytokines, and immune modulators, said methods comprising contacting, for example, an immortalized myeloma cell and a primary B cell or T cell in the presence of any one or more of the above-described proteins. Immortalized cells contemplated for use herein include human or mouse B cell myeloma cells, T cell myelomas, and the like, and antibody-synthesizing cells contemplated for use herein include purified spleen cells from an immunized mammal, and the like.

In accordance with a still further embodiment of the present invention, there are provided methods for the production of liposome-liposome fusions or liposome-cell fusions, said methods comprising contacting lipids suitable for the formation of liposomes and a suitable cell in the presence of one or more proteins as described herein.

In accordance with yet another embodiment of the present invention, there are provided improved methods for the intracellular delivery of bioactive compounds employing liposomes, the improvement comprising incorporating into said liposome one or more proteins as described herein.

The ability to promote efficient membrane fusion has broad applicability in clinical, industrial, and basic research situations. The reovirus fusion proteins could be used as alternatives to chemically-induced membrane fusion to promote cell-cell fusion, for example, during the production of hybridoma cells for monoclonal antibody production. In this instance, the reovirus fusion proteins would be inducibly expressed from inside a transiently or permanently transfected cell population to trigger fusion of these cells with a target cell population.

The atypical reovirus fusion proteins also have application in enhancing liposome-cell fusion. Liposomes have been developed as a means to introduce nucleic acids, proteins, and metabolic regulators into cells. Although liposome-cell fusion has been amply demonstrated, the unfavourable thermodynamics of membrane fusion contribute to variable efficiencies of fusion and cytotoxicity which lead to the development of proteoliposomes—liposomes containing specific proteins to promote cell binding and fusion.

Most of the proteoliposome studies reported in the art relate to the use of various enveloped virus fusion proteins. In accordance with the present invention, it is possible to take advantage of the novel structural features associated with the invention reovirus fusion proteins for use in proteoliposomes to enhance the intracellular delivery of bioactive compounds (e.g., nucleic acids, proteins or peptides, pharmacological agents, and the like), both in cell culture and in vivo.

The reovirus fusion proteins described herein promote membrane fusion in a diversity of cell types (e.g., fibroblasts and macrophages) from different species (e.g., avian and mammalian, including human) suggesting limited cell receptor-specificity as well as the general applicability of these proteins. It may also be possible to target reovirus fusion protein-containing proteoliposomes to specific cell types by including specific receptor-binding proteins in the liposome membrane. In this instance, the receptor-binding protein would confer targeted cell attachment of the liposome followed by subsequent enhanced liposome-cell fusion mediated by the reovirus fusion protein.

The demonstrated ability of P11 and P15 to induce cell-cell fusion indicates their potential use in the production of heterokaryons, for example, the generation of hybridomas for monoclonal antibody production. The induction of cell-cell fusion is usually triggered using the chemical fusogen polyethylene glycol (PEG). Although this procedure does trigger cell-cell fusion, toxic effects on cells hamper the efficiency of heterokaryon isolation. It is generally believed that “natural” membrane fusion is mediated by protein-lipid interactions, therefore, protein-mediated membrane fusion is likely to be much less cytotoxic than chemically-induced cell fusion.

The demonstrated ability of the small reovirus fusion proteins to promote efficient cell-cell fusion indicates their potential use as alternatives to chemical-induced cell fusion. Expression of P11 or P15 inside one population of cells, under the control of a strong inducible promoter, could trigger fusion with a second cell population, resulting in decreased cytotoxicity and more efficient heterokaryon isolation.

The atypical group of nonenveloped virus fusion proteins described herein represent alternatives to the use of enveloped virus fusion proteins in the protein-mediated enhancement of liposome-cell fusion for the intracellular delivery of bioactive molecules. The potential advantages of the reovirus fusion proteins relate to their unique structural and biological features. From a structural perspective, the small size and absence of N-linked glycosylation in the reovirus fusion proteins are the most apparent advantages offered by this system. The size, post-translational glycosylation, and complex tertiary structure of the enveloped virus fusion proteins makes synthesis and purification of the functional protein using recombinant DNA approaches and prokaryotic or eukaryotic expression systems problematic.

The majority of studies relating to the use of enveloped virus fusion proteins in proteoliposomes involve the production of virus particles which are subsequently purified, solubilized with detergent, and the viral envelopes containing the fusion protein are reconstituted into “virosomes” by removal of the detergent (see Grimaldi in Res. Virol., 146:289-293 (1995) and Ramani et al., FEBS Lett., 404:164-168 (1997)). Unlike most of the enveloped virus fusion proteins, the reovirus fusion proteins are small, nonglycosylated membrane proteins. Their small size and lack of N-linked glycosylation suggests that these proteins will be easier and more economical to produce in a functional form, without the concern of ensuring proper post-translational modification, using a diversity of expression and purification protocols. It is also likely that the small size of the reovirus fusion proteins contributes to less complex protein folding pathways and tertiary structure required for correct protein conformation. As a result, an increased diversity of extraction and solubilization procedures (e.g., choice of detergents and denaturants) should be available to facilitate purification of the functional fusion protein and incorporation into liposomes. In addition, the sequence of the reovirus fusion proteins reveals no obvious signal peptide required to promote co-translational membrane insertion. Hence, these proteins appear to be capable of signal peptide-independent post-translational membrane insertion, unlike enveloped virus fusion proteins. The ability to insert into membranes in a translation-independent manner offers a major advantage for the incorporation of these fusion proteins into liposome membranes.

The attractive biological properties of the reovirus fusion proteins relate to their immunogenicity and pH-independent fusion mechanism. The observation that these small reovirus fusion proteins are relatively non-immunogenic has profound implications for their use to promote liposome-cell fusion in vivo. An effective host immune response against any protein incorporated into the liposome membrane to promote cell fusion has adverse consequences. At the simplest level, a neutralizing antibody reponse to the fusion protein would contribute to decreased efficacy of the delivery system. More severe immune sequelae could involve humoral or cell-mediated immune recognition of cells containing the fusion protein in their cell membrane following successful liposome-cell fusion. The strongly immunogenic nature of enveloped virus fusion proteins makes these adverse consequences a significant possibility following administration of enveloped virus fusion protein-containing proteoliposomes, a situation unlikely to apply to the use of reovirus fusion proteins. Finally, the reovirus fusion proteins function at neutral pH, unlike the influenza virus HA protein, simplifying their use in cell culture and in vivo under physiological conditions.

In order to use reovirus fusion proteins for heterokaryon production, the proteins will need to be expressed in a controlled, inducible manner from within cells using standard recombinant DNA approaches. The utility of this approach has already been demonstrated in homologous cell-cell fusion in a non-inducible manner. In a similar fashion, these proteins can promote cell-cell fusion between heterologous cell types in an inducible manner.

The development of reovirus fusion proteins for enhanced liposome-cell fusion requires the expression and purification of the functional fusion proteins and their incorporation into liposome membranes to produce proteoliposomes. The P11 and P15 proteins can be expressed and purified using standard procedures. Expression can be accomplished employing a variety of expression systems, e.g., baculovirus or yeast eukaryotic expression vectors or from prokaryotic expression vectors, depending on expression levels and functional activity of the protein. Various detergent extraction procedures can be used to solubilize the proteins, which can then be purified as detergent-protein complexes using standard protein purification protocols. It may be necessary to try several different detergents to determine which are effective in solubilizing the protein while maintaining fusion activity. The small size and absence of N-linked glycosylation in the reovirus fusion proteins suggest that protein solubilization and purification should be considerably more simple than similar approaches to purify larger, more complex membrane proteins.

The detergent-protein complexes can be mixed with lipids and the detergent removed by dialysis, chromatography, or extraction according to standard published procedures, similar to methods used to generate influenza HA or Sendai virus F protein-containing virosomes (see Grimaldi, Res. Virol., 146:289-293 (1995) and Ramani et al., FEBS Lett., 404:164-168 (1997)). These procedures will result in the production of proteoliposomes, lipid vesicles containing the ARV, NBV, or BRV fusion proteins embedded in the vesicle membrane. Once again, optimal conditions for proteoliposome production can be empirically determined as can the lipid composition and size of the proteoliposomes which can affect the efficiency of liposome-cell fusion. Bioactive molecules of interest (e.g., nucleic acids, proteins or peptides, pharmacological compounds, and the like) can be included during the formation of the proteoliposmes to facilitate packaging of the molecule within the liposomes. The proteoliposomes can be purified by centrifugation and used to deliver bioactive molecules intracellularly, either in cell culture or in vivo, by protein-enhanced fusion of the proteoliposomes with cell membrances.

As acknowledged above, the use of liposomes or proteoliposomes for intracellular delivery of compounds is known in the art, and development of such methodology is proceeding on several fronts. What is unique with the present system is the use of an atypical, previously unidentified group of nonenveloped virus fusion proteins to promote membrane fusion. The unusual structural and functional properties of this new group of fusion proteins suggest that these proteins may circumvent many of the problems associated with the current development of protein-mediated membrane fusion.

The present invention also contemplates therapeutic compositions containing a physiologically tolerable carrier together with a fusion protein, polypeptide fragment thereof, or anti-fusion protein antibody, as described herein, dissolved or dispersed therein as an active ingredient. In a preferred embodiment, the therapeutic composition is not immunogenic when administered to a mammal or human patient for therapeutic purposes.

As used herein, the terms “pharmaceutically acceptable”, “physiologically tolerable” and grammatical variations thereof, as they refer to compositions, carriers, diluents, and reagents, are used interchangeably and represent that the materials are capable of administration to a mammal without the production of undesirable physiological effects such as nausea, dizziness, gastric upset, and the like.

Methods for the preparation of a pharmacological composition that contains active ingredients dissolved or dispersed therein is well known in the art. Typically such compositions are prepared as injectables either as liquid solutions or suspensions; however, solid forms suitable for solution, or suspension, in liquid prior to use can also be prepared. The preparation can also be emulsified.

The active ingredient can be mixed with excipients that are pharmaceutically acceptable and compatible with the active ingredient and in amounts suitable for use in the therapeutic methods described herein. Suitable excipients are, for example, water, saline, dextrose, glycerol, ethanol, or the like, as well as combinations thereof. In addition, if desired, the composition can contain minor amounts of auxiliary substances such as wetting or emulsifying agents, pH buffering agents, and the like, which enhance the effectiveness of the active ingredient.

The therapeutic composition of the present invention can include pharmaceutically acceptable salts of the components therein. Pharmaceutically acceptable nontoxic salts include the acid addition salts (formed with the free amino groups of the polypeptide) that are formed with inorganic acids such as, for example, hydrochloric acid, hydrobromic acid, perchloric acid, nitric acid, thiocyanic acid, sulfuric acid, phosphoric acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid, and the like.

Salts formed with the free carboxyl groups can also be derived from inorganic bases such as, for example, sodium hydroxide, ammonium hydroxide, potassium hydroxide, and the like; and organic bases such as mono-, di-, and tri-alkyl and -aryl amine (e.g., triethylamine, diisopropyl amine, methyl amine, dimethyl amine, and the like), and optionally substituted ethanolamines (e.g., ethanolamine, diethanolamine, and the like).

Physiologically tolerable carriers are well known in the art. Exemplary liquid carriers are sterile aqueous solutions that contain no materials other than the active ingredients and water, or contain a buffer such as sodium phosphate at physiological pH value, physiological saline, or both, such as phosphate-buffered saline. Still further, aqueous carriers can contain more than one buffer salt, as well as salts such as sodium and potassium chlorides, dextrose, polyethylene glycol, and other solutes.

Liquid compositions can also contain liquid phases in addition to and to the exclusion of water. Exemplary of such additional liquid phases are glycerin, vegetable oils such as cottonseed oil, and water-oil emulsions.

A therapeutically effective amount is a predetermined amount calculated to achieve the desired effect. The required dosage will vary with the particular treatment and with the duration of desired treatment; however, it is anticipated that dosages between about 10 micrograms and about 1 milligram per kilogram of body weight per day will be used for therapeutic treatment. In some instances, it may be particularly advantageous to administer such compounds in depot or long-lasting form. A therapeutically effective amount is typically an amount of a fusion protein according to the invention, or polypeptide fragment thereof that, when administered in a physiologically acceptable composition, is sufficient to achieve a plasma concentration of from about 0.1 μg/ml to about 100 μg/ml, preferably from about 1.0 μg/ml to about 50 μg/ml, more preferably at least about 2 μg/ml and usually 5 to 10 μg/ml. Antibodies are administered in proportionately appropriate amounts in accordance with known practices in this art.

The invention will now be described in greater detail by reference to the following non-limiting examples.

Two avian reovirus (ARV) strains were analyzed; strain 176 (see Hieronymous et al., Avian Dis., 27:255-259 (1983)) and strain 138 (see Drastini et al., Can. J. Vet. Res., 58:75-78 (1994)). The only known fusogenic mammalian reoviruses, i.e., Nelson Bay virus (NBV) (see Gard and Compans, supra) and baboon reovirus (BRV) (see Duncan et al. (1995), supra) were also analyzed. The genomes of these viruses have never been previously cloned or sequenced.

### EXAMPLE 1

### Virus Growth and Purification

The two strains of ARV were grown in monolayers of QM5 cells, a continuous quail cell line (see Antin and Ordahl, Devel. Biol., 143:111-121 (1991)) while the fusogenic mammalian reoviruses were grown in monkey Vero cells. Virus particles were isolated and concentrated from infected cell lysates by differential centifugation, as previously described (see Duncan, Virology, 219:179-189 (1996)).

### EXAMPLE 2

### Synthesis and Cloning of cDNA

The viral genomic dsRNA segments were isolated from concentrated virus stocks pretreated with RNase and DNase to remove extra-virion contaminating cellular nucleic acids. Virus particles were disrupted using 1% SDS and the viral dsRNA isolated by phenol-chloroform extraction and ethanol precipitation. Aliquots of genomic dsRNA (20 μg) were poly-A-tailed using E. coli poly-A polymerase, the tailed RNA was fractionated by agarose gel electrophoresis, and individual genomic segments were isolated using the RNaid protocol (Bio101) according to the manufacturers specified procedure. The tailed S class genome segments were used as templates for reverse transcription, using Superscript reverse transcriptase (Life Technologies Inc.) and an oligo-dT primer. Aliquots of the plus and minus strand cDNAs were used as templates for PCR amplification using Vent polymerase (New England Biolabs) and an oligo-dT primer containing a NotI restriction enzyme site. The products of the PCR reaction were digested with NotI, size-fractionated on agarose gels, and products corresponding to the full length S genome segments were gel-purified using Geneclean (Bio101). The individual, NotI-digested, double-stranded cDNAs were cloned into the NotI site of pBluescript (Stratagene) and used as templates for sequencing.

### EXAMPLE 3

### Sequencing and Sequence Analysis

The cloned cDNAs were sequenced using an automated DNA sequencer (Licor) at the NRC/Dalhousie Joint Sequencing Core Facility. All sequences were determined in their entirity from both cDNA strands. The full length cDNA sequences were compiled and analyzed using the GCG sequence analysis software (see Devereaux et al., Nucleic Acids Res., 12:387-395 (1984)).

### EXAMPLE 4

### Transfection and Cell Fusion Analysis

The ARV and NBV S1 cDNA clones and the BRV S4 cDNA clone were subcloned into the eukaryotic expression vector pcDNA3 (Invitrogen) under the control of the CMV promoter. Plasmid DNA was isolated and purified on Qiagen midi columns (Qiagen) according to the manufacturer's specifications. Plasmid DNA (1 μg) was mixed with Lipofectamine (3 μl) (Life Technologies Inc.) and used to transfect sub-confluent cell monolayers grown in 12 well cluster plates. Transfected cell monolyaers were incubated at 370° C. for 24-48 hr before being fixed with methanol and stained using a water-soluble Wright-Giemsa stain (DiffQuik; VWR-Canlab) or by immunostaining using viral-specific antiserum obtained from infected animals, as previously described (see Duncan et al., Virology, 224:453-464 (1996)). Cell fusion was assessed by light microscopy of stained monolayers and syncytial foci were photographed at 100× magnification.

### EXAMPLE 5

### Subcloning and Analysis of the Fusion-inducing Genome Segment

Sequence analysis determined that the ARV and NBV S1 genome segments contained three sequential overlapping open reading frames (ORFs) while the BRV S4 genome segment contained 2 ORFs. In order to determine which ORF encoded the viral fusion protein, portions of these genome segments were subcloned into pcDNA3 by PCR amplification of indivdual regions using sequence-specific primers as indicated in the figures. The subcloned regions were analyzed for their fusion-inducing ability by transfection analysis as described above.

### EXAMPLE 6

### Cloning and Sequencing the Reovirus Fusion Genes

Two unrelated fusion proteins responsible for the cell-cell fusion induced by avian reovirus (ARV) and the only two fusogenic mammalian reoviruses, Nelson Bay virus (NBV) and baboon reovirus (BRV) have been identified. These proteins are referred to herein as P11 (for ARV and NBV) and P15 (for BRV) to reflect their approximate predicted molecular weights. The genes encoding P11 from two strains of ARV (strain 176 and strain 138) have been cloned and sequenced, as has the gene from NBV that encodes P15. The sequence-predicted structural organization of these proteins has been analyzed, and the membrane fusion properties thereof have been directly demonstrated.

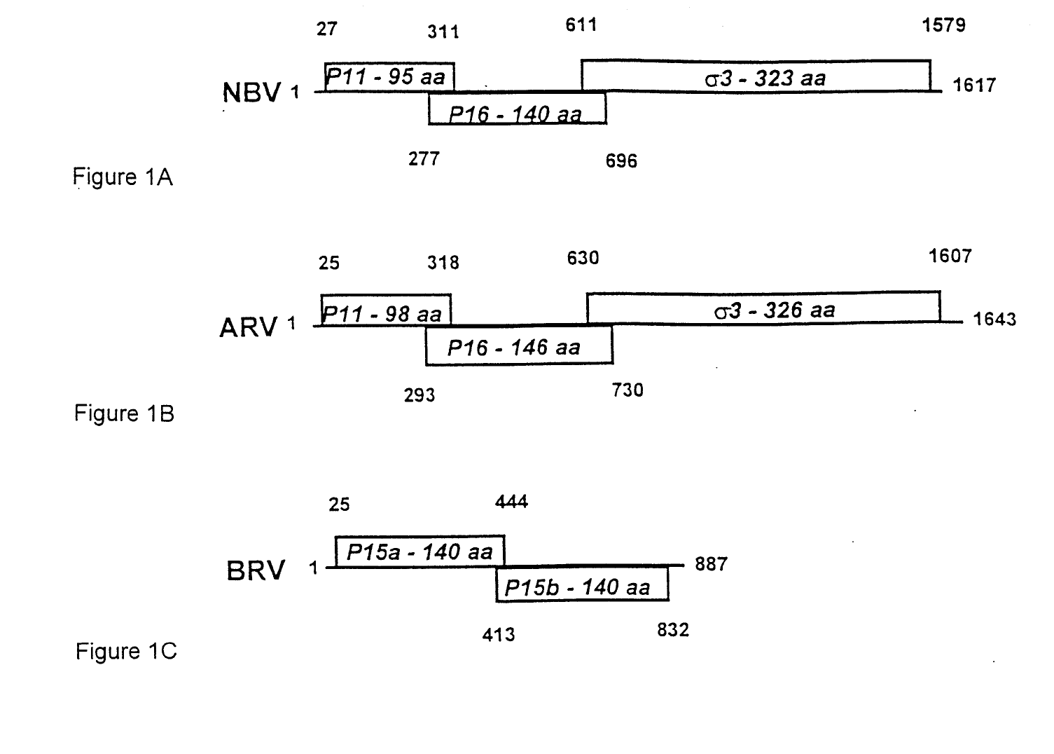

The ARV strain 138 and strain 176 sequences are highly homologous, exhibiting 96% amino acid identity in the predicted sequence of the P11 fusion protein. As a result, the following discussion of the ARV sequences refers to both strain 138 and strain 176. The ARV and NBV fusion proteins are encoded by the S1 genome segment of each virus. The organization of the ARV and NBV S1 genome segments and the analogous BRV S4 genome segment is indicated diagramatically in FIG. 1 and the cDNA sequences and predicted translation products are shown in SEQ ID Nos:1-10.

The S1 genome segment is 1643 or 1617 base pairs (bp) long for ARV and NBV, respectively, and appears to be functionally tricistronic, encoding three proteins from separate, sequential, overlapping open reading frames (ORFs). The 3′-terminal ORF encodes the s3 protein, the viral cell attachment protein and the only previously recognized product of the S genome segment (Varella and Benavente, J. Virol., 68:6775-6777 (1994); Shapouri et al., J. Gen. Virol., 76:1515-1520 (1995); Shapouri et al., J. Gen. Virol., 77:1203-1210 (1996); Theophilos et al., Virology, 208:678-684 (1995)). One unconfirmed report suggested that sigma3 represented the viral fusion protein (Theophilos et al. (1995), supra), although it has since been shown that this is incorrect (see Example 8).

The central ORF encodes a predicted 140-146 amino acid protein (referred to as P16) that has not been identified, as yet, and which shows no significant homology to any previously reported protein. The 5′-terminal ORF, encoding the P11 protein, begins at the first methionine codon and extends for 98 or 95 codons (ARV or NBV, respectively). Previous unpublished sequences obtained from the S1 genome segments of two Australian strains of ARV indicated a similar genetic organization (Kool and Holmes, Genbank submission). The sequence of the S1 genome segment of a third ARV isolate (strain S1133) showed a similar organization, although the first two ORFs were truncated, encoding 81 or 37 amino acids, respectively (Shapouri et al. (1995), supra). No sequences have previously been reported for NBV. Neither of the previous reports on the ARV S1 genome segment recognized the functional significance of the P11 ORF, concentrating instead on the s3-encoding ORF. Prior to the present invention, there has been no disclosure or suggestion in the prior art of any role of P11 of ARV or NBV in reovirus-induced cell fusion.

The BRV functional equivalent of the ARV and NBV S1 genome segments is the S4 genome segment which is approximately half the size (887 bp) of the S1 genome segments (see SEQ ID NO:9). The BRV S4 genome segment contains two sequential overlapping ORFs, each encoding 140 amino acid proteins (termed P15a and P15b). Although there is no sequence homology between either of these predicted gene products and the ARV or NBV P11 proteins, sequence analysis of P15a detected a predicted transmembrane domain suggesting that this protein possesses membrane interaction potential and may represent the fusion protein of BRV, a hypothesis that has been confirmed experimentally (see Example 8).

### EXAMPLE 7

### Sequence Analysis of the Reovirus Fusion Proteins

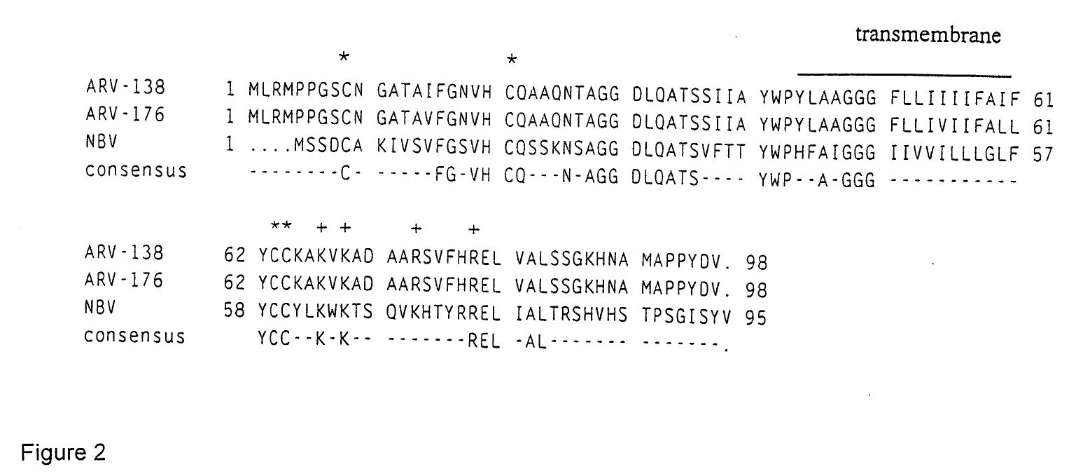

The ARV and NBV P11 proteins are small proteins (98 or 95 amino acids, respectively) that share approximately 38% sequence homology and a similar domain organization indicating that these proteins are evolutionarily related (FIG. 2). Both proteins lack obvious signal peptides, suggesting that they insert in membranes post-translationally. Both proteins also contain one predicted transmembrane domain located in the central portion of the protein resulting in small (approximately 40 amino acid) intracellular and extracellular domains. The conserved clustering of positively charged amino acids on the carboxy-proximal side of the transmembrane domain is consistent with the amino-terminal domain residing extracellularly (von Heijne, Curr. Op. Cell Biol., 2:604-608 (1990)). The four cysteine residues in each protein are conserved, suggesting that the ARV and NBV P11 proteins assume a similar tertiary and quaternary structure. The ARV P11 protein is devoid of N-linked glycosylation sites, implying that post-translational glycosylation is not required for functional protein folding, a prediction that has been confirmed experimentally (see Duncan et al. (1996), supra). Although the NBV P11 protein contains a single potential N-linked glycosylation site, this site is probably not glycosylated since inhibitors of glycosylation fail to affect NBV-induced cell fusion (see Wilcox and Compans (1983)). The size, absence of signal peptides, and N-linked glycosylation, and predicted domain organization of the ARV and NBV P11 proteins clearly distinguishes these proteins from the well characterized enveloped virus fusion proteins and suggests that P11 represents a novel type of membrane fusion protein.

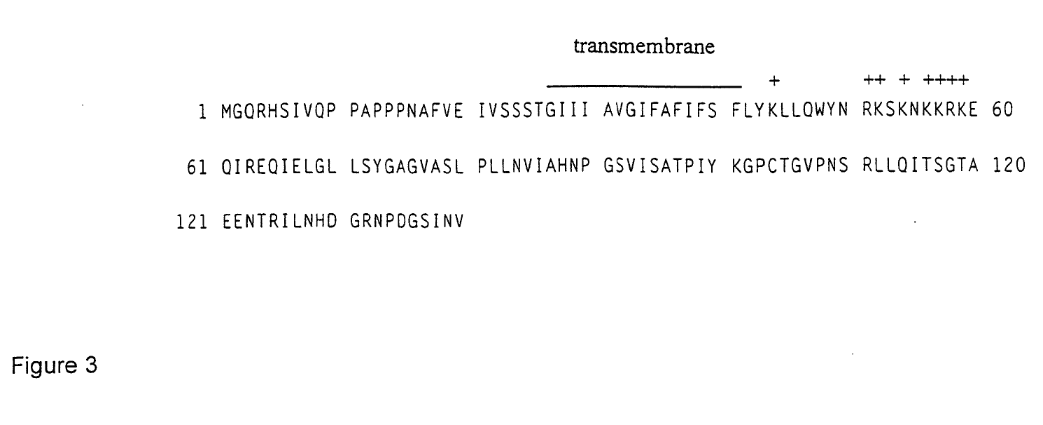

The BRV P15 fusion protein shares no obvious sequence homology with the ARV or NBV proteins and, at 140 amino acids, is considerably larger than the P11 proteins. The P15 protein has a predicted transmembrane domain with a clustering of positively charged amino acids on the carboxy side of this domain, suggesting that P15 is oriented with the amino-terminus of the protein external to the membrane, similar to the situation with ARV and NBV P11 (FIG. 3). However, P15a has a smaller external domain than P11 (25 amino acids, not 43), a much larger predicted internal domain (approximately 97 amino acids, not 37), and lacks the conserved cysteine residues of P11. The sequence and structural predictions of P15a suggest that this protein is unrelated to the NBV and ARV P11 proteins and thus, represents a second novel type of nonenveloped virus fusion protein.

### EXAMPLE 8

The P11 and P15 fusion proteins induce cell-cell fusion

The fusion-inducing potential of these reovirus proteins has been directly demonstrated by expressing them in transfected cells in the absence of any other reovirus proteins; intracellular expression triggers the induction of cell-cell fusion and syncytium formation characteristic of virus infection by this group of fusogenic reoviruses. Thus, quali cell monolayers were mock transfected, or transfected with plasmid DNA expressing the ARV, BRV, or NBV fusion proteins. Transfected cells were fixed and the nuclei stained using a Wright-Giemsa stain at 36 hr post infection and the stained monolayers were photographed at 100× magnification.

Transfection of plasmids expressing either ARV, NBV P11, or BRV P15 resulted in extensive cell fusion and the development of multinucleated syncytia (polykaryons). The appearance of polykaryons was evident when transfected cells were stained to display the cell nuclei, which clearly showed the clustering of nuclei within large syncytial cells. Mock transfected cells showed no signs of syncytium formation, indicating that cell fusion was the direct result of the expression of the reovirus proteins within transfected cells. These results conclusively demonstrate the membrane fusion-inducing capability of the reovirus fusion proteins of the invention.

Transfection of other reovirus proteins, including sigma3, which was previously hypothesized to represent the ARV fusion protein (see Theophilos et al. (1995), supra), fails to induce cell fusion, indicating that this is a P11- or P15-specific event. Furthermore, optimizing the ARV P11 translation start site to increase expression of this protein results in enhanced fusion activity while small deletions in P11 abrogate syncytium formation confirming that P11 alone is responsible for ARV-induced membrane fusion. In addition, all three proteins induce cell fusion in a variety of cell types of avian or mammalian origin indicating the general utility of these proteins to induce membrane fusion.

While the invention has been described in detail with reference to certain preferred embodiments thereof, it will be understood that modifications and variations are within the spirit and scope of that which is described and claimed.

### Summary of Sequences

SEQ ID NO:1 is a nucleotide sequence encoding a P11 protein obtained from Avian Reovirus strain 176 S1 (ARV1).

SEQ ID NO:2 is the deduced amino acid sequence of the P11 protein encoded by nucleotides 25-318 set forth in SEQ ID NO:1.

SEQ ID NO:3 is the deduced amino acid sequence of the P16 protein encoded by nucleotides 293-730 set forth in SEQ ID NO:1.

SEQ ID NO:4 is the deduced amino acid sequence of the sigma3 protein encoded by nucleotides 630-1607 set forth in SEQ ID NO:1.

SEQ ID NO:5 is a nucleotide sequence encoding a P11 protein obtained from ARV strain 138 S1 (ARV2).

SEQ ID NO:6 is the deduced amino acid sequence of the P11 protein encoded by nucleotides 25-318 set forth in SEQ ID NO:5.

SEQ ID NO:7 is the deduced amino acid sequence of the P16 protein encoded by nucleotides 293-730 set forth in SEQ ID NO:5.

That which is claimed is:

1. A protein characterized as:
having a molecular weight of about 11,000,
having less than about 100 amino acid residues,
having one transmembrane domain,
having a relatively small intracellular domain,
having a relatively small extracellular domain,
wherein said extracellular domain contains an amphipathic alpha helix motif, and
being relatively non-immunogenic,
wherein said protein is further characterized as lacking:
signal peptide, and
N-linked glycosylation signals.

2. A protein according to claim 1 having an amino acid sequence substantially the same as set forth in SEQ ID NO: 2 [ARV1], SEQ ID NO: 6 [ARV2] or SEQ ID NO: 8 [NBV].

3. A protein according to claim 1 having the amino acid sequence set forth in SEQ ID NO: 2 [ARV1], SEQ ID NO: 6 [ARV2] or SEQ ID NO: 8 [NBV].

4. A protein characterized as:
having a molecular weight of about 15,000,
having less than about 150 amino acid residues,
having one transmembrane domain,
having one relatively small intracellular domain,
having a relatively small extracellular domain,
wherein said extracellular domain contains an alpha helix motif, and
being relatively non-immunogenic,
wherein said protein is further characterized as lacking:
signal peptide, and
N-linked glycosylation signals.

5. A protein according to claim 4 having an amino acid sequence substantially the same as set forth in SEQ ID NO 10 [BRV].

6. A protein according to claim 4 having the amino acid sequence set forth in SEQ ID NO: 10 [BRV].

7. An antibody raised against the protein of claim 1.

8. An antibody raised against the protein of claim 4.

9. An isolated nucleic acid encoding protein according to claim 1.

10. An isolated nucleic acid according to claim 9 having a contiguous nucleotide sequence substantially the same as:
nucleotides 25-1607 of SEQ ID NO: 1 [ARV1],
nucleotides 25-1607 of SEQ ID NO: 5 [ARV2],
nucleotides 27-1579 of SEQ ID NO: 7 [NBV], or
variations thereof which encode the same amino acid sequence, but employ different codons for some of the amino acids, or splice variant nucleotide sequences thereof.

11. An isolated and purified nucleic acid, or functional fragment thereof encoding the protein of claim 1, selected from:
(a) DNA encoding the amino acid sequence set forth in SEQ ID NO: 2, SEQ ID NO: 6 or SEQ ID NO: 8, or
(b) DNA that hybridizes to the DNA of (a) under moderately stringent conditions, wherein said DNA encodes biologically active fusion protein, or
(c) DNA degenerate with respect to either (a) or (b) above, wherein said DNA encodes biologically active fusion protein.

12. An isolated nucleic acid according to claim 9 operatively associated with an inducible promoter.

13. An isolated nucleic acid encoding protein according to claim 4.

14. An isolated nucleic acid according to claim 13 having a contiguous nucleotide sequence substantially the same as:
nucleotides 25-832 of SEQ ID NO: 9 [BRV], or
variations thereof which encode the same amino acid sequence, but employ different codons for some of the amino acids, or splice variant nucleotide sequences thereof.

15. An isolated and purified nucleic acid, or functional fragment thereof encoding the protein of claim 4, selected from:
(a) DNA encoding the amino acid sequence set forth in SEQ ID NO: 10, or
(b) DNA that hybridizes to the DNA of (a) under moderately stringent conditions, wherein said DNA encodes biologically active fusion protein, or
(c) DNA degenerate with respect to either (a) or (b) above, wherein said DNA encodes biologically active fusion protein.

16. An isolated nucleic acid according to claim 13 operatively associated with an inducible promoter.

17. Cells containing protein according to claim 1.

18. Cells containing protein according to claim 4.

19. Cells containing nucleic acid according to claim 9.

20. Cells containing nucleic acid according to claim 12.

21. Cells containing nucleic acid according to claim 13.

22. Cells containing nucleic acid according to claim 16.

23. Liposomes containing protein according to claim 1.

24. Liposomes containing protein according to claim 4.

25. Liposomes containing nucleic acid according to claim 9.

26. Liposomes containing nucleic acid according to claim 13.

27. A method for the production of protein according to claim 1, said method comprising expressing nucleic acid encoding said protein in a suitable host.

28. A method for the production of protein according to claim 4, said method comprising expressing nucleic acid encoding said protein in a suitable host.

29. A method to promote membrane fusion, said method comprising contacting the membranes to be fused with an effective amount of a protein according to claim 1.

30. A method according to claim 29 wherein said membranes are cell membranes, liposome membranes or proteoliposome membranes.

31. A method to promote membrane fusion, said method comprising contacting the membranes to be fused with an effective amount of a protein according to claim 4.

32. A method according to claim 31 wherein said membranes are cell membranes, liposome membranes or proteoliposome membranes.

33. A method for the production of B cell or T cell hybridoma cells useful for the production of monoclonal antibodies, cytokines, immune modulators, or other heterokaryons, said method comprising contacting an immortalized myeloma cell and a primary B cell or T cell in the presence of a protein according to claim 1.

34. A method according to claim 33 wherein said immortalized cell is an human or mouse B cell myeloma cell or a T cell myeloma, wherein said antibody-synthesizing cell is a purified spleen cell from an immunized mammal.

35. A method for the production of hybridoma cells useful for the production of monoclonal antibodies, said method comprising contacting an immortalized cell and an antibody-synthesizing cell in the presence of a protein according to claim 4.

36. A method according to claim 35 wherein said immortalized cell is an human or mouse B cell myeloma cell or a T cell myeloma, wherein said antibody-synthesizing cell is a purified spleen cell from an immunized mammal.

37. A method for the production of liposome-liposome fusions or liposome-cell fusions, said method comprising contacting lipids suitable for the formation of liposomes and a suitable cell in the presence of a protein according to claim 1.

38. A method for the production of liposome-liposome fusions or liposome-cell fusions, said method comprising contacting lipids suitable for the formation of liposomes and a suitable cell in the presence of a protein according to claim 4.

39. In a method for the extracellular or intracellular delivery of bioactive compounds employing liposomes, the improvement comprising incorporating into said liposome a protein according to claim 1.

40. In a method for the extracellular or intracellular delivery of bioactive compounds employing liposomes, the improvement comprising incorporating into said liposome a protein according to claim 4.

41. An isolated nucleic acid fragment useful as a hybridization probe, wherein said fragment comprises at least 14 contiguous nucleotides of the nucleic acid according to claim 9, and wherein said fragment is labeled with a detectable substituent.

42. An isolated nucleic acid fragment useful as a hybridization probe, wherein said fragment comprises at least 14 contiguous nucleotides of the nucleic acid according to claim 13, and wherein said fragment is labeled with a detectable substituent.


SEQUENCE LISTING (15 sequences)

SEQ ID NO: 1 - DNA, 1643 bp, avian reovirus strain 176
  CDS (25)...(318): nucleotide sequence encoding P11 protein (SEQ ID NO2)
gctttttcaatcccttgttcgtcgatgctgcgtatgcctcccggttcgtgtaacggtgcgactgctgtatttggtaacgttcattgtcaggcagctcaaaacacggcaggtggtgatttgcaagctacgtcatccataattgcatattggccttatctagcggcgggtggtggtttcttattaattgttatcattttcgctcttctatactgttgtaaggctaaggtcaaggcggacgctgcacgtagtgtcttccatcgtgagctggtagcgttgagttctggtaagcacaatgcaatggctccgccatacgacgtttgaagtgcaacgatttaatttctgtccgctatcacttcgcgaacttgctatcccatcatttactgctataactggggctgacccatcacagtattttaacattgagctcccacacactcatcctctctattccaaattgcctactctgttatctcaaccttgtagggtccacgtgcggctgattcgccggttcgctctctattcaacattgtcaagtatttgtgagtacgattgtgctctactattctccccacacgctatcgttccattgcctgcatccgatcggcggtcttgtcttatagttcattgggatggcgggtctcaatccatcgcagcgaagagaggtcgtcagcttgatactgtcattgacttcgaacgtgactataagtcatggcgatttgacgccgatctatgaacggctgaccaatctagaagcgtctacggagttattacatcgctccatttccgatatatccactactgtctcaaatatttctgcaaatttacaagacatgacccataccttggatgatgtaactgctaatttagacggtttgaggaccactgttactgcacttcaggattccgtctccattctgtctacaaatgtgactgacttaacgaacacatcctctgcgcacgcggcgacactatcttcacttcaaactacggttgacggaaactccactgccatctccaatttgaagagtgatgtatcgtcgaacggtttagctattacagatctgcaggatcgtgttaaatcattggagtctaccgcgagtcatggtctatctttttcgcctccgcttagtgtcgctgacggcgtggtttcattagacatggacccctacttctgttctcaacgagtttctttaacatcatactcggcggaggctcaactaatgcaatttcggtggatggcacggggtactaacggatcatctgataccattgacatgaccgttaacgctcactgtcatggaagacgcactgattatatgatgtcgtccacgggaaatctcacggtcactagtaacgtcgtgttattaaccttcgatttaagtgacataacgcatatcccatcagacctagcacgtcttgttcccagtgcgggattccaagctgcgtcgttccctgtggacgtatcattcacccgcgattctgcgactcatgcgtaccaagcgtatggggtgtactcgagctcacgtgtcttcacaattactttcccaaccggaggtgatggtacagcgaacattcgttccttgaccgtgcgtaccggcatcgacacctaaggtgtggcgccgtactgggattggttattcatc

SEQ ID NO: 2 - protein, 98 aa, avian reovirus strain 176
MLRMPPGSCNGATAVFGNVHCQAAQNTAGGDLQATSSIIAYWPYLAAGGGFLLIVIIFALLYCCKAKVKADAARSVFHRELVALSSGKHNAMAPPYDV

SEQ ID NO: 3 - protein, 146 aa, avian reovirus strain 176
MQWLRHTTFEVQRFNFCPLSLRELAIPSFTAITGADPSQYFNIELPHTHPLYSKLPTLLSQPCRVHVRLIRRFALYSTLSSICEYDCALLFSPHAIVPLPASDRRSCLIVHWDGGSQSIAAKRGRQLDTVIDFERDYKSWRFDADL

SEQ ID NO: 4 - protein, 326 aa, avian reovirus strain 176
MAGLNPSQRREVVSLILSLTSNVTISHGDLTPIYERLTNLEASTELLHRSISDISTTVSNISANLQDMTHTLDDVTANLDGLRTTVTALQDSVSILSTNVTDLTNTSSAHAATLSSLQTTVDGNSTAISNLKSDVSSNGLAITDLQDRVKSLESTASHGLSFSPPLSVADGVVSLDMDPYFCSQRVSLTSYSAEAQLMQFRWMARGTNGSSDTIDMTVNAHCHGRRTDYMMSSTGNLTVTSNVVLLTFDLSDITHIPSDLARLVPSAGFQAASFPVDVSFTRDSATHAYQAYGVYSSSRVFTITFPTGGDGTANIRSLTVRTGIDT

SEQ ID NO: 5 - DNA, 1643 bp, avian reovirus strain 138
  CDS (25)...(318): nucleotide sequence encoding P11 protein (SEQ ID NO6)
gctttttcaatcccttgtttgtcgatgctgcgtatgcctcccggttcgtgtaacggtgcaacagctatctttggtaacgtccattgtcaggcggctcaaaatactgccggcggcgacttgcaagctacctcatccataattgcctattggccttatctagcggcgggtggtggttttttgttgattattattatttttgccatcttctactgttgtaaggctaaagttaaagcggacgctgcacggagtgttttccaccgtgagcttgtagcactgagctctggtaagcacaatgcaatggctccgccatacgacgtttgaagtgcaacgctttgatttctgcccaatatcacttcgtgagcttgccaccccatcgtttactgctataattgggattgacccatcacgttattttaatattgagctttcgcacacgcatcctctctactctaagttgccgactctgttatcgcagccctgccgagtccacgtgcgtttgattcgtagattcgctctctgttcaacgctgtcgagtatctgcgagtacgattgtgcgttactactttccccacacgccatcactccactgtcctcatccgatcagcgatcttatcttatagttcattgggatggcgggtctcaatccatcacagcgaagagaggtcgtcagcttgatactgtcattgacttcgaacgcgcatataaatcatggcgatttgacgccaatctatgaacggttgaccagtttagaagcgtctgcggaatcactatatcgctccatttccagcatgtctactaccgtttcagacatttcagcagatttgcagaacgtgactcgcgccttggatgatgtgactgctaatttagatggtatgagagtcaccattactacgcttcaagattctgtgtccactctctcaacgactgtaactgatttaacaaacacctcttctgtgcactcggaagcactgtcttcactccgaactatagttgatgggaactccactaccattgataatttgaaaagtgatgtatcatcaaacggtcttgctatcacagacctgcagagtcgtgttaaatccttggaatctgtttcgagtcacgggctatctttttcgcctcctcttagtgtcgctgacgacgtagtgtcgttgagtatggacccttacttttgctctcagcgagtcaccttgacatcatactcagcagaagctcaactgatgcaattccaatggatggcaagaggtgctaacggatcatcagacactattgacatgaccgtcaatgctcactgtcatgggagacgcactgattacataatgtcgtccacgggaggtcttacagttactagtaatgccgtgtctttaaccttcgacttgagttacattacacgcctcccaccagacctctcgcgtcttgttcccagtgcaggattccaagccgcgtcgttccccgtggatgtatccttcaccagagattcgacaactcatacatatcaagcttatggagtgtattctagttcgcgtgtatttaccatcactttcccgactggtggtgacggtcccgcaaatatccgtttcctaaccgtgcgtaccggcatcgacacctaaggtgtggcgccgtacggggattggttattcatc

SEQ ID NO: 6 - protein, 98 aa, avian reovirus strain 138
MLRMPPGSCNGATAIFGNVHCQAAQNTAGGDLQATSSIIAYWPYLAAGGGFLLIIIIFAIFYCCKAKVKADAARSVFHRELVALSSGKHNAMAPPYDV

SEQ ID NO: 7 - protein, 146 aa, avian reovirus strain 138
MQWLRHTTFEVQRFDFCPISLRELATPSFTAIIGIDPSRYFNIELSHTHPLYSKLPTLLSQPCRVHVRLIRRFALCSTLSSICEYDCALLLSPHAITPLSSSDQRSYLIVHWDGGSQSITAKRGRQLDTVIDFERAYKSWRFDANL

SEQ ID NO: 8 - protein, 326 aa, avian reovirus strain 138
MAGLNPSQRREVVSLILSLTSNAHINHGDLTPIYERLTSLEASAESLYRSISSMSTTVSDISADLQNVTRALDDVTANLDGMRVTITTLQDSVSTLSTTVTDLTNTSSVHSEALSSLRTIVDGNSTTIDNLKSDVSSNGLAITDLQSRVKSLESVSSHGLSFSPPLSVADDVVSLSMDPYFCSQRVTLTSYSAEAQLMQFQWMARGANGSSDTIDMTVNAHCHGRRTDYIMSSTGGLTVTSNAVSLTFDLSYITRLPPDLSRLVPSAGFQAASFPVDVSFTRDSTTHTYQAYGVYSSSRVFTITFPTGGDGPANIRFLTVRTGIDT

SEQ ID NO: 9 - DNA, 1617 bp, Nelson Bay virus
  CDS (27)...(311): nucleotide sequence encoding P11 protein (SEQ ID NO10)
gcttaattttgctcttgggtgtcgatatgagtagtgactgtgccaaaattgtctctgtgtttgggagtgtgcattgccagtcttctaagaattcggctggtggagatcttcaggcgacatccgttttcacgacctattggccacattttgccattggtgggggtattatagtagtaatcttgttgcttggactattctattgctgttatcttaagtggaagacatcccaggtcaagcacacgtatcgtcgtgagctaatagcccttactcgtagtcatgtccattcaaccccatctggtatttcgtatgtgtgagagttctttttatgagccttgggtgcgatctggttacagatctgagattagtttcatttgccgtcgtgagttaacgtattatattaacgtgcacattcctttagaccatccacaacgttcagtcgcttgcgctctatctcagacccccgttgcttggcacgtgtctttgcttcgtcgtcgatcgtacgacccatcacttccggatttttgtgagcttgactgtgtgctgcggcacattcgtccaatcccgagaagattggtgtctcgaggtttctcctctcacgttgtcgttcactatgacagaaccactcagtccccagcagcgaaaagaggttgtagccttgattttgacgatgaaccagagcataagcgcttcgcgatctgacatgagtgcgctcgagaagcgagtgtctatcattgaatcagcgcaggctgctttacgtgtcgatgttacttctttgcagtcagttagttccggattgaattccaccatgcacgatctgtcagcgtctgtcgcgaatctcaagactatcgtcaatactatgtcgtcaacagttgccactatggaaggtgaattgcaaagttgtaagagtgagatttctaacacgcaaaatgtactgtcagttgtacagacagagctgagcaatgcgcaatctggattagcatccatgacgactagcttgtcaaacttaacgactagtgtgaacgctaacgctgtggccatatctggactcaaagcctctcttaactcactgtctagctcaattcctacatcactcgcatctcccctgactgtctcaggcggtattttaagtctgtctatgaatcgtaaattttgtggtgacgctgctggtttaaattcatattccacattgtcccagatgcagtcctttaactcgaatgttccaacgtcattatctggtaccaatctgtccacttctattcttgtgcattcgcgtggtggtttgactgtattcaatttgtctacgactcatgctttcacacctacgtcggttgataccaaattgactatcgactgtcgaacttttaccccgtctccaagtgattggtccgttctaataccaaaaccagcatttcaatcgagcaattttctgtgtacgggttggatgtgtgtcaacgacgcatggatcccggcaagtgtgatcggtgcggtggatagtaatcctaaggtcatgttcttgcatctgactacgcggccttcacagcgaattacgggcttggtcatctatttctctatcgacacgtagggggtggctcccaccactaagagatgctactcatc

SEQ ID NO: 10 - protein, 95 aa, Nelson Bay virus
MSSDCAKIVSVFGSVHCQSSKNSAGGDLQATSVFTTYWPHFAIGGGIIVVILLLGLFYCCYLKWKTSQVKHTYRRELIALTRSHVHSTPSGISYV

SEQ ID NO: 11 - protein, 140 aa, Nelson Bay virus
MSIQPHLVFRMCESSFYEPWVRSGYRSEISFICRRELTYYINVHIPLDHPQRSVACALSQTPVAWHVSLLRRRSYDPSLPDFCELDCVLRHIRPIPRRLVSRGFSSHVVVHYDRTTQSPAAKRGCSLDFDDEPEHKRFAI

SEQ ID NO: 12 - protein, 323 aa, Nelson Bay virus
MTEPLSPQQRKEVVALILTMNQSISASRSDMSALEKRVSIIESAQAALRVDVTSLQSVSSGLNSTMHDLSASVANLKTIVNTMSSTVATMEGELQSCKSEISNTQNVLSVVQTELSNAQSGLASMTTSLSNLTTSVNANAVAISGLKASLNSLSSSIPTSLASPLTVSGGILSLSMNRKFCGDAAGLNSYSTLSQMQSFNSNVPTSLSGTNLSTSILVHSRGGLTVFNLSTTHAFTPTSVDTKLTIDCRTFTPSPSDWSVLIPKPAFQSSNFLCTGWMCVNDAWIPASVIGAVDSNPKVMFLHLTTRPSQRITGLVIYFSIDT

SEQ ID NO: 13 - DNA, 887 bp, baboon reovirus
  misc_feature (25)...(444): CDS
gtaaatttttctcgcgataagtacatgggtcaaagacattcaatagttcaaccaccagctccaccgccaaatgcttttgttgaaattgtgagcagttctactggcattataatcgctgttggcatatttgcatttatattctcatttttatataagttgctgcagtggtacaatcgtaagtcaaagaataagaaacgtaaagagcaaattagagaacaaattgagcttggtttattatcatatggtgctggagtagcatcacttcctttgctcaacgttattgcacataatcctggatcagttatctcggctacccctatctataaaggtccgtgcactggtgtacctaattcgcgcctacttcaaatcacgagcgggactgcagaagagaacactagaattttgaatcatgatggaagaaacccagatggaagtatcaacgtttgagtggccaaagtcattagatgaaagtttgcaagtgttatgtaatgagttgaagggaaagactgaatggcaagatgacatggaagattggatgccatactggatatatatgaaacatgatggtattgctatctcgcaatccagatactcactactccagcaactagctgtatgggtgtggaagtgcttcgactttgatatgtgtgtgtacaatatctggacgacatggttagtaaaacatgcatgttctcgatgtcctgagttcgatgatgaggccttctggtctggggtgccaacaattattaaattagtaattaggaagacaatgcataggtacgcttatcttgatgatagtactcttgcggatttgactgagcaggttgggctctgagttcattgaccatgtagagactgcatgcacgcagcgcggaaagtcattcatc

SEQ ID NO: 14 - protein, 140 aa, baboon reovirus
MGQRHSIVQPPAPPPNAFVEIVSSSTGIIIAVGIFAFIFSFLYKLLQWYNRKSKNKKRKEQIREQIELGLLSYGAGVASLPLLNVIAHNPGSVISATPIYKGPCTGVPNSRLLQITSGTAEENTRILNHDGRNPDGSINV

SEQ ID NO: 15 - protein, 140 aa, baboon reovirus
MEETQMEVSTFEWPKSLDESLQVLCNELKGKTEWQDDMEDWMPYWIYMKHDGIAISQSRYSLLQQLAVWVWKCFDFDMCVYNIWTTWLVKHACSRCPEFDDEAFWSGVPTIIKLVIRKTMHRYAYLDDSTLADLTEQVGL